(19) United States
(12) Patent Application Publication
(10) Pub. No.: US 2008/0139962 A1
(43) Pub. Date: Jun. 12, 2008

(54) Analyte Collection Apparatus and Method

(76) Inventors: Ahmed Jehanli, Oxfordshire (GB); Dene Baldwin, Oxfordshire (GB)

Correspondence Address: NIXON & VANDERHYE, PC, 901 NORTH GLEBE ROAD, 11TH FLOOR, ARLINGTON, VA, 22203, US

(21) Appl. No.: 11/792,945

(22) PCT Filed: Dec. 13, 2005
(86) PCT No.: PCT/GB05/50241

§ 371 Date: Jun. 13, 2007

(30) Foreign Application Priority Data
Dec. 14, 2004 (GB) 0427240.7

(51) Int. Cl. A61B 10/00; B65D 69/00

(52) U.S. Cl. 600/573; 206/569

(57) ABSTRACT

An analyte collection apparatus is provided, comprising a gripper; and a compressible analyte collecting portion releasably gripped by the gripper, wherein the gripper exerts a force to grip and compress the analyte collecting portion. The analyte collecting portion can be immersed in a buffer solution whilst in the partially compressed state, and allowed to expand therein, reducing the time required for non-specific binding of analytes to the analyte collecting portion. The analyte collection device facilitates indirect handling of the collected analyte, preventing cross-contamination, and also allows for quick on-site testing.

### FIELD OF THE INVENTION

The present invention relates to an analyte collection apparatus and method, and more particularly, though not necessarily, to an analyte collection apparatus and method for gathering oral fluid samples.

### BACKGROUND OF THE INVENTION

Body fluids are useful for the detection of analytes of diagnostic value and for therapeutic drug monitoring. Traditionally, blood and urine have been the matrices of choice for diagnostic testing. Recently, interest in saliva (oral fluid) testing has gained momentum. Many analytes such as antibodies, antigens, hormones, pharmaceutical drugs and drugs of abuse are present in saliva. Oral fluid can also be used as a source of DNA material for genetic testing. The main advantages of oral fluid over other biological fluids are: collection is non-invasive, painless, does not require training and it can be carried out under observation. Oral fluid is already being used in laboratory-based and point-of-care tests for the detection of drugs of abuse, viruses and bacteria like HIV and Helicobacter pylori, therapeutic drug monitoring and DNA typing. Oral fluid is now being proposed as the preferred and most convenient matrix for testing motorists driving under the influence of drugs.

There are two ways for collecting oral fluid, direct expectoration in a container and, more commonly, placing an absorbent pad in the mouth. The pad can be made of cellulose, cotton or rayon material. Oral fluid is then extracted from the pad either by centrifugation or compression. A number of prior devices for the collection of oral fluid have been developed.

U.S. Pat. No. 4,768,238 describes a simple device for collecting saliva by expectoration into a cup. This method is not suitable for someone with a “dry mouth” syndrome. US2004087874 describes the use of flavouring-coated Pasteur pipette for aspirating oral fluid from the cavity under the tongue. This is not suitable for someone with dry mouth and the pipette can get blocked with any debris in the mouth cavity. Furthermore, use of flavouring material to stimulate salivation can result in marked drop in the levels of many analytes, e.g., cocaine. A more sophisticated method described in U.S. Pat. No. 6,652,481 and U.S. Pat. No. 6,022,326 by Lifepoint Inc. uses plastic tubing connected to a vacuum pump to aspirate oral fluid from the mouth cavity. Again, this procedure is not suitable for someone with a dry mouth. It also faces the possibility of blockage of the device.

EP0418739 and U.S. Pat. No. 5,335,673 by Epitope Inc., describe the use of an absorbent pad attached to a stick for the collection of oral fluid. This is then placed in a container and sent to the laboratory for extraction and testing of the sample. The absorbent pad is coated with a mixture of dried salt, detergent, proteins like albumin and gelatin and preservatives in order to minimize stickiness of analytes to the pad. This device does not allow on-site testing as sample extraction requires centrifugation. The use of additives in the absorbent pad can affect the level of some analytes. Persons to be tested would also object to having such a mixture of foreign material placed in their mouth. A similar device is described in EP0734686 A1. An absorbent pad has a built-in sealing arrangement. The pad is placed in the mouth to collect saliva, sealed and then sent to the laboratory for sample extraction and testing. Again, this device does not provide a sample suitable for on-site testing.

Orasure Inc.'s device (WO 03/028889) has a piece of foam attached to a compressible holder. The foam end is placed in the mouth to collect saliva. Oral fluid is then expressed from the foam by squeezing it into a special container that is coupled with a lateral flow test strip. This device is not suitable for collecting oral fluid for analysis by methods other than lateral flow.

U.S. Pat. No. 5,526,0031 by Saliva Diagnostic Systems Inc. describes a saliva collection system consisting of a nitrocellulose pad with a volume adequacy indicator. The saliva collected by the pad (1 mL) is extracted from the pad into a suitable buffer using mechanical filtration (Serum filter tube, described in U.S. Pat. No. 5,786,227). This device has the disadvantage that it may take up to 40 minutes to obtain a sufficient sample from individuals with dry mouth. This is not acceptable if the device is to be used for road-side testing.

One of the original methods for collecting saliva is that developed by Sarstedt (U.S. Pat. No. 4,774,962). A piece of absorbent pad made of foam or a cellular material is chewed by a person then placed into a centrifuge tube for extracting the saliva. This procedure is not suitable for on-site testing and there are serious implications with regard to hygiene as the pad has to be directly handled. Similarly, the device described by Trinity Research Ltd. (WO9418891 and EP0056241) uses a piece of cotton pad to collect saliva. This is then placed into a special tube. Saliva is extracted by compressing the pad by means of screw type mechanism. This device also raises questions of hygiene as the pad requires handling to place in the mouth of the person.

We have recently described an oral fluid device (EP1389445) that uses a polyurethane type foam to collect relatively large volume of oral fluid (>1.0 mL) for laboratory testing. The device was also designed to couple with a lateral flow type on-site test.

A number of patents that are grouped together (US20044171173, US2002146346, US56440087, U.S. Pat. No. 6,303,081, US20020004019, U.S. Pat. No. 6,248,598, US2004019301 and WO9950656) describe a variety of integrated systems that combine saliva collection with a lateral flow type on-site test. These devices are not suitable for collecting saliva for other types of tests.

### SUMMARY OF THE INVENTION

According to a first aspect of the present invention, there is provided an analyte collection apparatus as defined in the appended claim 1. Further aspects and embodiments of the invention are defined in the other appended claims.

The apparatus of the present invention may be used, for example, to collect oral fluid for diagnostic testing. The device uses an absorbent pad for collecting saliva. It allows for a hygienic and rapid collection of sample (<1 minute), immediate extraction of the oral fluid for both on-site and laboratory-based testing and a procedure for ensuring optimal recovery of analytes from the absorbent material. The device may also be used for other biological matrices as well as solid material.

### BRIEF DESCRIPTION OF THE DRAWINGS

FIG. 1 shows a fully assembled analyte collection apparatus in accordance with an embodiment of the invention;

FIG. 2 shows the separated components of the apparatus of FIG. 1;

FIG. 3 illustrates how to open the apparatus of FIG. 1; and

FIG. 4 illustrates a testing kit in accordance with a further embodiment of the invention.

### DETAILED DESCRIPTION OF THE INVENTION

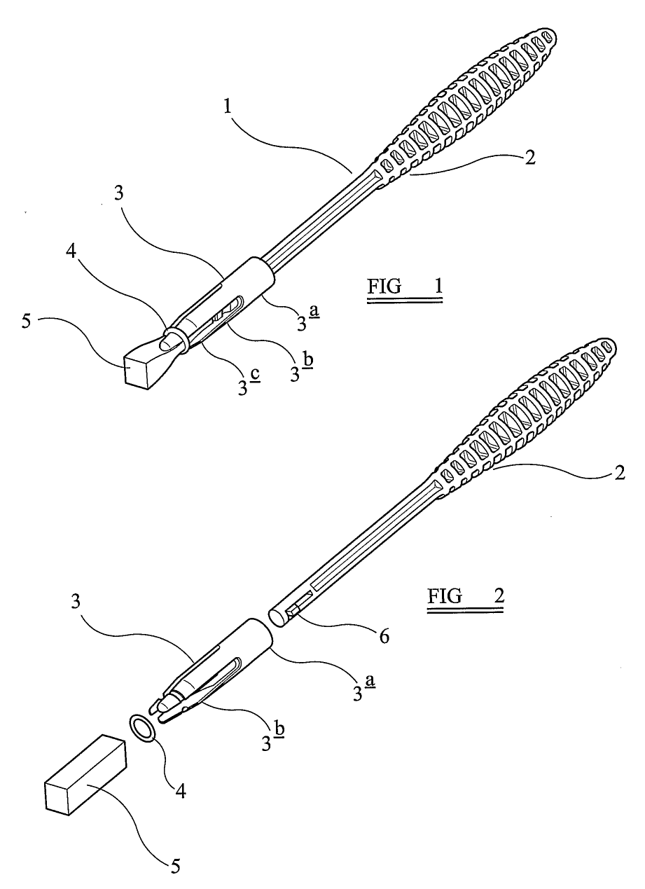

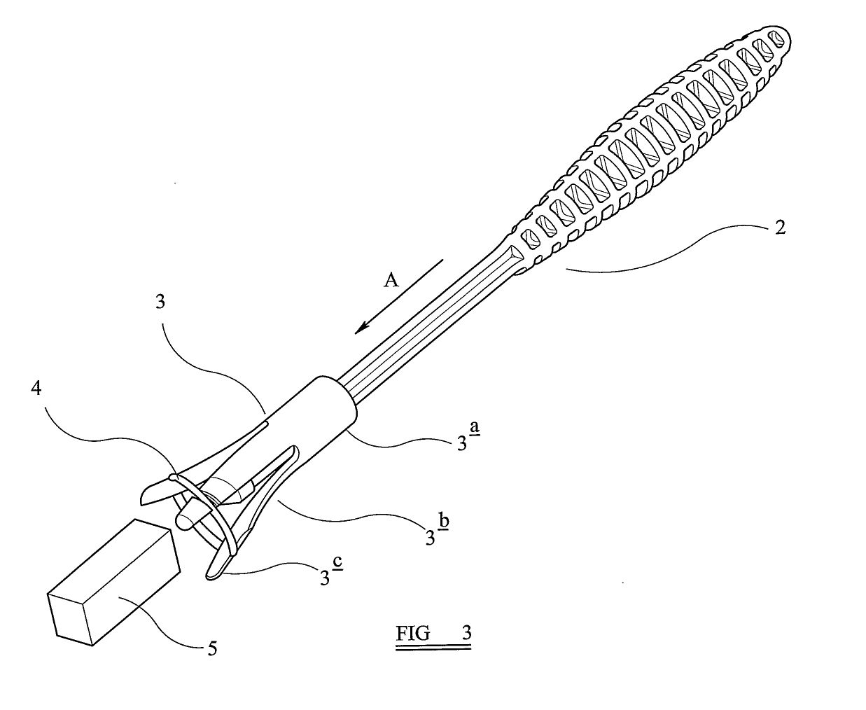

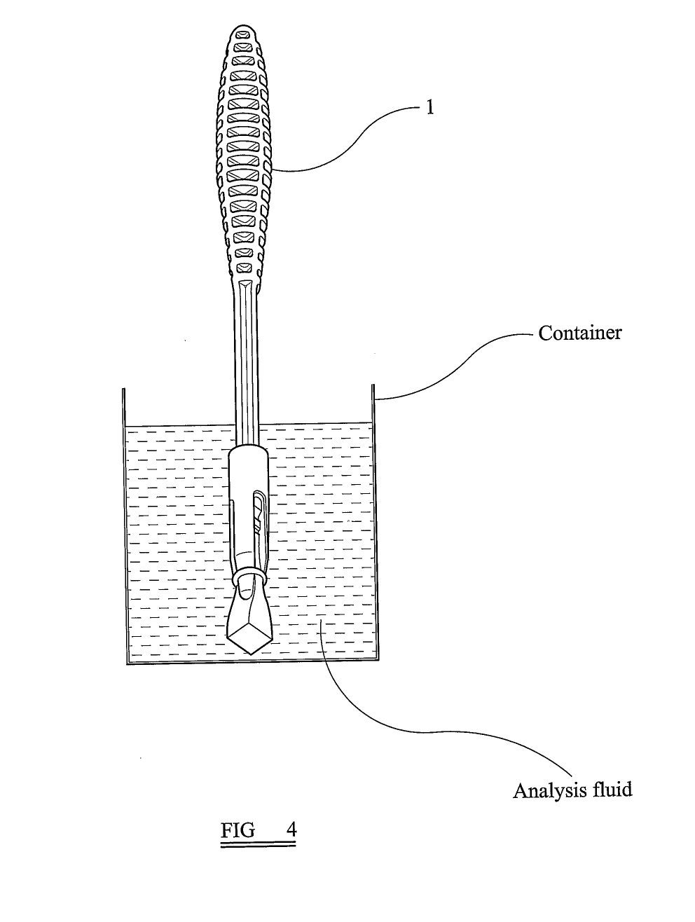

FIGS. 1 to 3 illustrate a fluid collection apparatus in accordance with an embodiment of the invention. The apparatus is shown in a closed configuration in FIG. 1, and in an open configuration in FIG. 3. FIG. 2 shows the components of the apparatus in an exploded view.

The fluid collection apparatus 1 comprises four parts: a handle 2 that is made of hardened plastic or any other appropriate material, a gripper 3 made of hard plastic that has a hollow cylindrical collar 3a for receiving a cylindrical end of the handle. The other end of the gripper 3 is molded to provide a collet 3b which comprises three splayed fingers 3c extending axially from the collar 3a and which open to receive an absorbent pad 5 for collecting fluid. The collet 3b is molded in such a way that a moderate force is required to close the fingers 3c about the pad 5. The collet is held in the closed position by an O-ring 4 which is retained in a groove within the arms 3c.

The apparatus is assembled by pushing the handle 2 into the gripper 3, forcing the arms 3c of the collet 3b to open against the resistance of the O-ring 4, as illustrated by the motion of the handle in the direction of arrow A in FIG. 3. A one-way latch 6 on the handle 2 snap fits into a groove (not shown) on the gripper to hold it in place, preventing accidental disengagement of the handle and gripper when reversing the handle. Placing the opened arms 3c around the pad 5 and reversing the direction of travel of the handle 2 will close the arms 3c and secure the foam 5 in place within the gripper. This has the effect of compressing the pad 5 to some extent. The volume of oral fluid collected depends upon the size of pad used. The pad may be made of foam. The foam used (10×5×5 mm) in one embodiment is made of polyurethane material (supplied by Rynel, USA). However, other materials known in the art can be used such as cotton or cellulose.

The actual collection procedure is as follows. The collector is placed in the mouth of the subject to collect oral fluid. This can be achieved by placing the pad in the side between the cheek and the gum for 30 seconds. Alternatively, the pad can be placed under the tongue. The pad can also be used to wipe the gum and tongue which is of particular importance if cellular material is needed for DNA testing. A combination of the three methods is also possible. The pad has the capacity to absorb up to 0.75 mL of sample. However, the action of the gripper 3b on the pad 5, holding it in an at least partially compressed state, restricts the volume collected to about 0.25-0.3 mL. The pad can be released from the device in two ways following removal from the subject's mouth. The gripper is placed in a suitable container as illustrated in FIG. 4, then held at its distal end 3a whilst the handle 2 is pushed forward (in the same way as described above with reference to FIG. 3). This causes the arms 3c of the collet 3b to open and the pad 5 to fall into the suitable container. Alternatively, the collector 1 may be placed vertically, foam-first into a container until the base of the container is reached. Continued downward movement of the handle 2 will cause the collet to open and the foam deposited in the container. Both procedures of operation are safe as the foam is not handled before or after collection of oral fluid.

The apparatus described here offers a novel way of reducing potential adhesion of analytes to the pad during analysis and allowing optimal release of analytes from the pad. In one application of the apparatus for testing for the presence of drugs of abuse in saliva, oral fluid is collected as described above. The end of the apparatus including the pad is placed into a bottle containing a suitable buffer solution (1 mL) designed for extracting drugs from the pad (Cozart Bioscience Product No. CR-BUFF) so that the pad is fully immersed in the buffer. The pad used has a capacity for 0.75 mL sample volume. However, this is restricted by the gripper to collecting about 0.25 mL. When the pad is released from the gripper in the buffer it quickly expands to its full capacity by absorbing part of the buffer. This action markedly reduces the time-dependent non-specific binding of drugs and other analytes to the pad. This is very important if the oral fluid sample were to be sent away for laboratory testing.

The analyte collection apparatus described above addresses many of the problems with known analyte collection devices. The gripping arrangement allows the pad to be handled indirectly, avoiding the risks of cross-contamination. The apparatus is small and can be used in conjunction with a small bottle of buffer solution to provide a convenient on-site fluid testing apparatus, thereby avoiding the need for centrifuges or other such equipment. The pad avoids the problem of acquiring blockages, and is quick to absorb fluid, especially oral fluid, even if the subject suffers from dry mouth.

The apparatus can be used to provide samples for “point-of-care” testing as is the case for road-side testing of motorists driving under the influence of drugs. Oral fluid is collected for 30 seconds as described above and the pad is deposited into a 7 mL plastic bottle containing 0.5 mL of extraction buffer. The oral fluid-buffer mixture is immediately expelled from the pad using a filter tube such as that described in U.S. Pat. No. 5,786,227 which forms a tight fit with the inner wall of the bottle. The filter tube is pushed down the bottle squeezing the pad and releasing the liquid sample into the tube. This can be used to run a lateral flow test for drugs of abuse.

Another application of the apparatus is for testing solid surfaces for contamination with drugs of abuse and for testing sweat for analytes. The pad can be slightly wetted with a suitable buffer and used to wipe the surface to be tested, for example the forehead or any other part of the body, to collect sweat. The collected sample can be extracted as described above for further testing.

A further application is in the field of detecting illicit powder or resinous material such as cocaine. The pad is slightly wetted with a suitable buffer then used to wipe the solid resin or to pick up powdered material. The drugs are then extracted as described above.

The apparatus can be sterilised by autoclaving then used as a swab to collect material from, for example, the throat, for identification of bacterial and viral infection by providing bacterial cells or viruses for culturing.

According to an alternative embodiment, the pad has an opening for receiving an end of a gripping apparatus. The apparatus is expanded within the pad to grip the pad and push it outwards, partially compressing the pad.

It will be obvious to persons skilled in the art that this apparatus has other application, for example, in the field of veterinary medicine and environmental pollutants testing.

It will be appreciated by the person skilled in the art that various modifications may be made to the above embodiments without departing from the scope of the present invention as set out in the accompanying claims.

1-14. (canceled)

15. An analyte collection apparatus, comprising
a compressible analyte collecting portion;
a gripper having a handle and a mouth that can be opened to receive the analyte collecting portion and closed to grip and compress the analyte collecting portion; and
biasing means for biasing the mouth towards a closed position.

16. Apparatus as claimed in claim 15, wherein the gripper comprises a gripping head which is slideably attached to the handle such that the handle and the gripping head can move axially relative to one another.

17. Apparatus as claimed in claim 16, wherein the gripping head comprises the mouth, and relative axial motion of the handle and the gripping head in a first direction causes the mouth to open, and relative axial motion in a second opposite direction causes the mouth to close.

18. Apparatus as claimed in claim 17, wherein the gripping head comprises a substantially cylindrical base portion mounted upon the handle, and a plurality of axially extending fingers for receiving and gripping the analyte collecting portion, the plurality of fingers comprising the mouth.

19. Apparatus as claimed in claim 18, wherein the biasing means comprises an O-ring disposed around the plurality of fingers.

20. Apparatus as claimed in claim 16, wherein one of the gripping head and the handle comprises a snap-latch for engaging the other to facilitate assembly of the two and to prevent their separation after assembly.

21. Apparatus as claimed in claim 15, wherein the device is suitable for oral use.

22. Apparatus as claimed in claim 15, wherein the analyte collecting portion comprises a liquid absorbing material.

23. Apparatus as claimed in claim 22, wherein the liquid absorbing material is a sponge.

24. A testing kit comprising apparatus as claimed in claim 15, and a container arranged to receive the analyte collecting portion.

25. A testing kit as claimed in claim 24, wherein the container contains analysis fluid.

26. A testing kit as claimed in claim 25, wherein the analysis fluid is a buffer solution.

27. A method of collecting analytes comprising the steps of:
receiving an analyte collecting portion within a gripping means;
closing the gripping means to compress the analyte collecting portion;
applying the analyte collecting portion to a region containing the analyte to be collected; and
releasing the analyte collecting portion into a container such that the analyte collecting portion expands to an uncompressed state in the container.

28. A method as claimed in claim 27, wherein the collected fluid is oral fluid.
